(19) United States
(12) Patent Application Publication
(10) Pub. No.: US 2021/0186578 A1
(43) Pub. Date: Jun. 24, 2021

(54) TRUMPET EXPANDABLE VERTEBRAL DEVICE

(71) Applicant: BioLife Medical Device Inc., HsinChu (TW)

(72) Inventors: Li-Sen Chen, Hsinchu (TW); Che-Yang Chao, Hsinchu (TW); Li-Hwan Kao, Hsinchu (TW)

(21) Appl. No.: 17/120,194

(22) Filed: Dec. 13, 2020

(30) Foreign Application Priority Data
Dec. 18, 2019 (TW) 108146387

(51) Int. Cl. A61B 17/70; A61F 2/46

(52) U.S. Cl. CPC A61B 17/7098; A61F 2002/30579; A61F 2/4611; A61F 2/4601

(57) ABSTRACT

A trumpet expandable vertebral device, includes: a center shaft having a front end and a rear end; a trumpet expandable part mounted outside and surrounding the center shaft, wherein the trumpet expandable part includes: plural expandable plates, each expandable plate having a thickness which increases from the front end toward the rear end; a fixed part, for fixing the relative position of the trumpet expandable part with respect to the center shaft in an axial direction of the center shaft, but allowing the center shaft to rotate with respect to the trumpet expandable part within the fixed part in a radial direction of the center shaft; and a connection part for connecting the trumpet expandable plates with the fixed part; a trumpet expanding ring mounted outside and surrounding the center shaft, wherein the trumpet expanding ring is located between the center shaft and the trumpet expandable part in the radial direction; wherein when the trumpet expanding ring moves toward the rear end relatively to the trumpet expandable part, the trumpet expandable plates are expanded to expand a vertebra.

### CROSS REFERENCE

The present invention claims priority to TW 108146387 filed on Dec. 18, 2019.

### BACKGROUND OF THE INVENTION

### Field of Invention

The present invention relates to a trumpet expandable vertebral device; particularly, it relates to such a trumpet expandable vertebral device which, when inserted into a human body, can radially expand itself so as to assist restoring a vertebra to its original shape.

### Description of Related Art

When a human spine (e.g. cervical vertebrae, thoracic vertebrae, lumbar vertebrae, etc.) suffers a compression fracture, or collapses due to poor bone mass density, an expandable vertebral device which is inserted into the spine can help to expand the space to restore the spine to a better condition. U.S. Pat. No. 7,846,206 B2, U.S. Pat. No. 9,414,933 B2, and TW 1589266 disclose different types of expandable vertebral devices.

In comparison with the prior arts, the present invention provides a different mechanical structure to reduce the number of required components, whereby the manufacture and assembly of the vertebral device is much easier.

### SUMMARY OF THE INVENTION

From one perspective, the present invention provides a trumpet expandable vertebral device comprising: a center shaft having a front end and a rear end; a trumpet expandable part mounted outside and surrounding the center shaft, wherein the trumpet expandable part includes: plural expandable plates, each expandable plate having a thickness which increases from the front end toward the rear end; a fixed part, surrounding and holding a predetermined portion of the center shaft, but allowing the center shaft to rotate with respect to the trumpet expandable part within the fixed part in a radial direction of the center shaft; and a connection part for connecting the trumpet expandable plates with the fixed part; a trumpet expanding ring mounted outside and surrounding the center shaft, wherein the trumpet expanding ring is located between the center shaft and the trumpet expandable part in the radial direction; wherein when the trumpet expanding ring moves toward the rear end relatively to the trumpet expandable part, the trumpet expandable plates are expanded to expand a vertebra.

In one embodiment, the center shaft has a main body and an exterior surface of the main body is threaded, and wherein an interior surface of the trumpet expanding ring is correspondingly threaded.

In one embodiment, the rear end of the center shaft is an operable end which is configured to be driven by a corresponding tool, such that the center shaft rotates within the fixed part and rotates relatively to the expandable plates.

In one embodiment, the operable end has a hexagonal shape.

In one embodiment, the trumpet expanding ring includes at least one protrusion which is configured to insert into a slit which is formed when the expandable plates are expanded, to be in contact with a side of at least one of the expandable plates.

In one embodiment, the center shaft includes a peripheral surface and the fixed part is mounted to press against the peripheral surface.

In one embodiment, the trumpet expandable vertebral device further comprises a nut which is connected to the front end of the center shaft.

In one embodiment, the center shaft comprises a hollow interior and is provided with an inlet and at least one outlet for the injection of a filler into a human body.

In one embodiment, the center shaft is provided with at least one outlet on a radial surface of the rotatable center shaft.

In one embodiment, each of the expandable plates has a width D, and the connection part is connected with each expandable plate by a width d, and d<(1/2)D.

The objectives, technical details, features, and effects of the present invention will be better understood with regard to the detailed description of the embodiments below, with reference to the attached drawings.

### BRIEF DESCRIPTION OF THE DRAWINGS

FIG. 1 shows a trumpet expandable vertebral device according to one embodiment of the present invention, in expanded state.

FIG. 2 shows the trumpet expandable vertebral device in FIG. 1, in disassembled state.

FIG. 3 shows the trumpet expandable vertebral device in FIG. 1, in closed state.

FIG. 4 shows a trumpet expandable vertebral device according to another embodiment of the present invention, in expanded state.

FIG. 5 shows the trumpet expandable vertebral device in FIG. 4, in disassembled state.

### DESCRIPTION OF THE PREFERRED EMBODIMENTS

The drawings as referred to throughout the description of the present invention are for illustration only, to show the interrelations between the components, but not drawn according to actual scale of shapes and sizes.

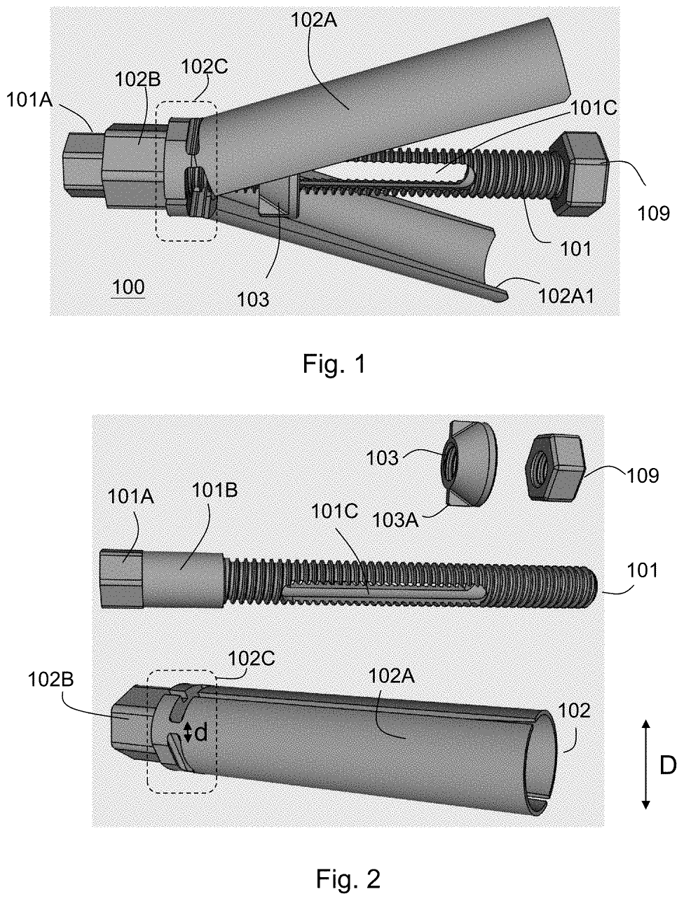

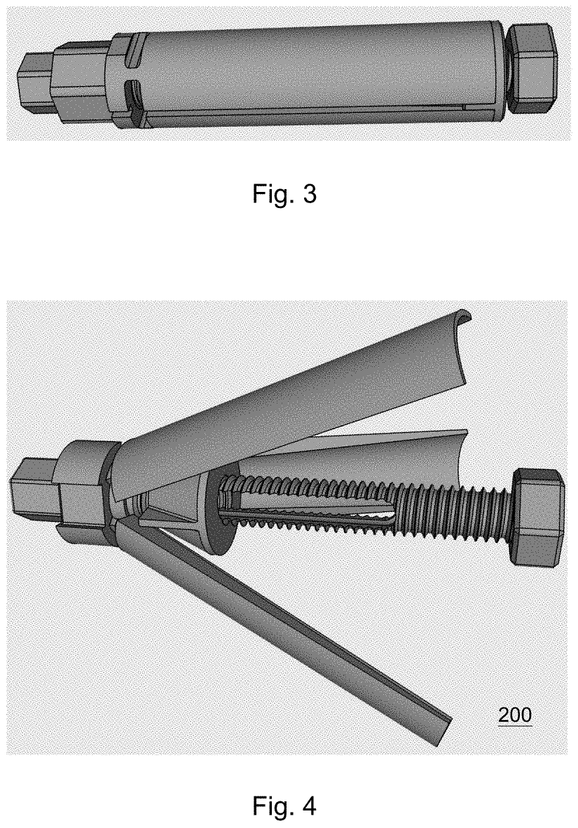

Please refer to FIGS. 1-3, which show a trumpet expandable vertebral device according to an embodiment of the present invention (i.e. trumpet expandable vertebral device 100), in its expanded state (FIG. 1), disassembled state (FIG. 2) and closed state, respectively. The right side, in the drawings, of the trumpet expandable vertebral device 100 is the front end and the left side of the trumpet expandable vertebral device 100 is the rear end. When an surgical operation is performed to insert the trumpet expandable vertebral device 100 into a human body, the right side enters the human body first.

The trumpet expandable vertebral device 100, according to the present invention, includes a center shaft 101, a trumpet expandable part 102 and a trumpet expanding ring 103. The trumpet expandable part 102 and the trumpet expanding ring 103 are mounted outside and surrounding the center shaft 101. By the term “mounted”, it means that the trumpet expandable part 102 and the trumpet expanding ring 103 surround and hold the center shaft 101, but it is not necessary for the trumpet expandable part 102 and the trumpet expanding ring 103 to be fixed to the center shaft 101. In a radial direction of the center shaft 101, the trumpet expanding ring 103 is located between the center shaft 101 and the trumpet expandable part 102. The center shaft 101 has a main body and an exterior surface of the main body is threaded, and an interior surface of the trumpet expanding ring 103 is correspondingly threaded. The rear end (left side) of the center shaft 101 is an operable end 101A; when an surgical operation is performed and the operable end 101A is activated to rotate, the center shaft 101 is driven to rotate within the trumpet expanding ring 103 and relatively to the trumpet expanding ring 103, whereby the center shaft 101 moves relatively to the trumpet expanding ring 103 along an axial direction of the center shaft 101 (that is, whereby the trumpet expanding ring 103 moves relatively to the center shaft 101 along the axial direction of the center shaft 101). In the embodiment as shown in FIG. 2, the operable end 101A has a hexagonal shape, and can be driven by a tool having a corresponding shape. However, the present invention can be embodied in many other ways, as long as the operable end 101A can be driven by a corresponding tool.

The trumpet expandable part 102 includes expandable plates 102A, a fixed part 102B and a connection part 102C. There are plural expandable plates 102A; each expandable plate 102A has a thickness 102A1 which increases from the front end (right side of the figure) toward the rear end (left side of the figure). This thickness variation is an important feature of the present invention and will be explained in more detail later below. In the shown embodiment, the expandable plates 102A have a rectangular shape in a projection surface which is parallel to the axial direction of the center shaft 101 and have an arc shape in another projection surface which is parallel to the radial direction of the center shaft 101, but this is only one example and the shape of the expandable plates 102A can be embodied in other forms. The fixed part 102B is configured to fix a relative position of the trumpet expandable part 102 with respect to the center shaft 101 in the axial direction of the center shaft 101, that is, so that the positional relationship between the trumpet expandable part 102 and the center shaft 101 is held stable. The relative position between the trumpet expandable part 102 and the center shaft 101 can be fixed by any feasible ways, and what is shown by the embodiment is one example. In this embodiment as shown in the drawings, the center shaft 101 includes a peripheral surface 101B and the fixed part 102B is mounted outside the peripheral surface 101B of the center shaft 101, to press against the operable end 101A. Thus, in the axial direction of the center shaft 101, the relative relationship between the trumpet expandable part 102 and the center shaft 101 is fixed relatively, while in the radial direction of the center shaft 101, the center shaft 101 is able to rotate within the fixed part 102B, relatively to the trumpet expandable part 102. The connection part 102C connects the expandable plates 102A with the fixed part 102B.

When an operator, such as a surgeon, drives the center shaft 101 to rotate relatively to the trumpet expanding ring 103 (which also is to rotate relatively to the trumpet expandable part 102), whereby the center shaft 101 move within and relatively to the fixed part 103 toward the right side, the trumpet expanding ring 103 can be driven toward the left side relatively to the trumpet expandable part 102. Because the thickness 102A1 of the expandable plate 102A is varied, such as constantly increasing from the right side of the figure toward the left side of the figure, when the trumpet expanding ring 103 is driven toward the left side relatively to the trumpet expandable part 102, the expandable plates 102A are expanded, achieving the function of restoring the height and shape of the vertebrae.

Note that “constantly increasing (from the right side of the figure toward the left side of the figure)” is only one embodiment of the present invention. Under the spirit of the present invention, the increase of the thickness can be arranged in many other ways. For example, in the shown embodiment, the thickness increase rate is substantially a constant, but in other embodiments, the thickness increase rate can be varied, or a certain portion of the expandable plate 102A can have a constant thickness. These variations can still achieve the objective for the expandable plate 102A to expand as the center shaft 101 rotates relatively to the trumpet expanding ring 103.

Please refer to FIG. 2. In one preferred embodiment, the trumpet expanding ring 103 includes at least one protrusion 103A, and more preferably, includes protrusions 103A having a number equal to the number of the expandable plates 102A. The protrusion 103A is configured to be inserted into a slit which is formed between the lateral edges of the expandable plates 102A when they are expanded, to be in contact with a side edge of a corresponding expandable plate 102A. Thus, when the expandable plates 102A tend to compress due to a resistance coming from human body, because the protrusion 103A is in contact with or limited by the edges of the expandable plate 102A, the trumpet expanding ring 103 can be held in the desired position and held not to be pushed backward according to the axial direction of the center shaft 101.

In one preferred embodiment, the center shaft 101 comprises a hollow interior and is provided with an inlet and at least one outlet for the injection of a filler into a human body during a surgical operation, so that when the trumpet expandable vertebral device 100 is inserted into the human body, the filler such as bone cement or bone graft can be injected from the inlet, through the hollow interior, to outflow from the outlet into the vertebrae. In one embodiment, the filler is injected form the inlet at the left side (at the operable end 101A) of the center shaft 101, through the hollow interior of the center shaft 101, to outflow out from the outlet at the right side of the center shaft 101. Referring to FIGS. 1 and 2, in a more preferable embodiment, the center shaft 101 is further provided with outlets (e.g. 101C) on its radial surface to multiply the channels for releasing the fillers, so that the filler injected form the inlet at the left side of the center shaft 101, in addition to flowing through the hollow interior of the center shaft 101 and flowing out from the outlet at the right side of the center shaft 101, also flows out in the radial direction from the radial outlets provided on the radial surface of the center shaft 101. The number, size and shape of the outlets 101C are shown as an example, not for limiting the scope of the present invention.

Please refer to FIG. 2, in one preferred embodiment, each expandable plate 102A has a width D (i.e. the perimeter of the expandable plate 102A in the radial surface), and the connection part 102C is connected with each expandable plate 102A by a width d, and d<D, to reduce the required force for expanding the expandable plate 102A. In a more preferable embodiment, d<(1/2)D.

In one preferred embodiment, a nut 109 is fitted to or is fixed to the right side of the center shaft 101, to prevent the trumpet expanding ring 103 from detaching from the center shaft 101. However, if this is not a concern, the nut 109 can be omitted.

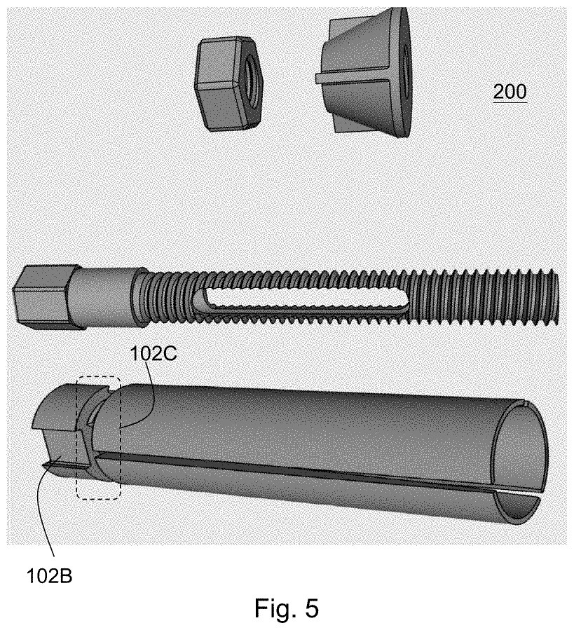

Please refer to FIGS. 4-5, which show a trumpet expandable vertebral device according to another embodiment of the present invention, in expanded state and in disassembled state, respectively. This embodiment shows that: the number of the expandable plates is not limited to two, but can be a number more than two; so is the number of the protrusions 103A of the trumpet expanding ring 103. Also, please refer to FIG. 5, the shapes and structures of the fixed part 102B and the connection part 102C are not limited to the shapes and structures as shown in FIGS. 1-3, but can be modified as desired; the spirit of the present invention is fulfilled as long as the fixed part 102B can assist to fix the relative relationship between the trumpet expandable part 102 and center shaft 101 in the axial direction of the center shaft 101, and the connection part 102C allows the expandable plate 102A to expand.

The present invention has been described in considerable detail with reference to certain preferred embodiments thereof. It should be understood that the description is for illustrative purpose, not for limiting the scope of the application of the present invention. An embodiment or a claim of the present invention does not need to be regarded as having to achieve all the objectives or advantages of the present invention. The title and abstract are provided for assisting searches but not for limiting the scope of the present invention. Those skilled in this art can readily conceive variations and modifications within the spirit of the disclosure in this specification, which should therefore be included in the scope of the following claim(s) made by the inventors. For example, as long as the primary objective is attained, other extra components can be added, or, components that are given separate reference numbers and described as separate items can be combined into one piece. As an example, the fixed part 102B and the connection part 102C can be manufactured in one piece, or be regarded as one single unit. As another example, the expandable plates do not necessary be of the shape as shown, that is, to have a rectangular shape in a projection on a surface which is parallel to the axial direction of the center shaft 101 and have an arc shape in a projection on a surface which is parallel to the radial direction of the center shaft 101; the expandable plates can have other shapes as long as they can be expanded by the relative rotation of the center shaft 101 within the trumpet expanding ring 103. It is not limited for each of the embodiments described hereinbefore to be used alone; under the spirit of the present invention, two or more of the embodiments described hereinbefore can be used in combination. For example, two or more of the embodiments can be used together, or, a part of one embodiment can be used to replace a corresponding part of another embodiment. In view of the foregoing, the spirit of the present invention should cover all such and other modifications and variations, which should be interpreted to fall within the scope of the following claims and their equivalents.

What is claimed is:

1. A trumpet expandable vertebral device, comprising:
a center shaft having a front end and a rear end;
a trumpet expandable part holding and surrounding the center shaft, wherein the trumpet expandable part includes:
plural expandable plates, each expandable plate having a thickness which increases from the front end toward the rear end;
a fixed part, for fixing a relative position of the trumpet expandable part with respect to the center shaft in an axial direction of the center shaft, but allowing the center shaft to rotate with respect to the trumpet expandable part within the fixed part in a radial direction of the center shaft; and
a connection part for connecting the trumpet expandable plates with the fixed part; and
a trumpet expanding ring mounted outside and surrounding the center shaft, wherein the trumpet expanding ring is located between the center shaft and the trumpet expandable part in the radial direction;
wherein when the trumpet expanding ring moves toward the rear end relatively to the trumpet expandable part, the trumpet expandable plates are expanded to restore the height and shape of a vertebra.

2. The trumpet expandable vertebral device of claim 1, wherein the center shaft has a main body and an exterior surface of the main body is threaded, and wherein an interior surface of the trumpet expanding ring is correspondingly threaded.

3. The trumpet expandable vertebral device of claim 1, wherein the rear end of the center shaft is an operable end which is configured to be driven by a corresponding tool, such that the center shaft rotates within the fixed part and rotates relatively to the expandable plates.

4. The trumpet expandable vertebral device of claim 3, wherein the operable end has a hexagonal shape.

5. The trumpet expandable vertebral device of claim 1, wherein the trumpet expanding ring includes at least one protrusion which is configured to insert into a slit which is formed when the expandable plates are expanded, to be in contact with a side of at least one of the expandable plates.

6. The trumpet expandable vertebral device of claim 1, wherein the center shaft includes a peripheral surface and the fixed part is mounted outside the peripheral surface.

7. The trumpet expandable vertebral device of claim 1, further comprising a nut which is connected to the front end of the center shaft.

8. The trumpet expandable vertebral device of claim 1, wherein the center shaft comprises a hollow interior and is provided with an inlet and at least one outlet for the injection of a filler into a human body.

9. The trumpet expandable vertebral device of claim 8, wherein the center shaft is provided with at least one outlet on a radial surface of the rotatable center shaft.

10. The trumpet expandable vertebral device of claim 1, wherein each of the expandable plates has a width D, and the connection part is connected with each expandable plate by a width d, and d<(1/2)D.
